(19) United States
(12) Patent Application Publication
(10) Pub. No.: US 2008/0194526 A1
(43) Pub. Date: Aug. 14, 2008

(54) Liquid Formulations

(76) Inventors: Vivian Georgousis, West Caldwell, NJ (US); Wei-Qin Tong, Basking Ridge, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/997,909

(22) PCT Filed: Aug. 8, 2006
(86) PCT No.: PCT/US2006/030836

§ 371 Date: Feb. 5, 2008

Related U.S. Application Data
(60) Provisional application No. 60/706,820, filed on Aug. 9, 2005.

(51) Int. Cl. A61K 31/66; A61K 31/131; A61P 37/00

(52) U.S. Cl. 514/114; 514/663

(57) ABSTRACT

Disclosed is a concentrate for dilution comprising a S1P receptor agonist or a pharmaceutically acceptable salt thereof, propylene glycol and optionally glycerin. This formulation is adapted for patients in a difficult condition to swallow.

The present invention relates to pharmaceutical compositions comprising a sphingosine-1 phosphate receptor modulator or agonist or a pharmaceutically acceptable salt thereof. Sphingosine-1 phosphate (hereinafter “S1P”) is a natural serum lipid. Presently there are eight known S1P receptors, namely S1P1 to S1P8. S1 P receptor modulators or agonists are typically sphingosine analogues, such as 2-substituted 2-amino-propane-1,3-diol or 2-amino-propanol derivatives, e.g. a compound comprising a group of formula X

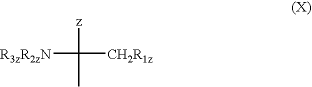

wherein Z is H, C₁₋₆alkyl, C₂₋₆alkenyl, C₂₋₆alkynyl, phenyl, phenyl substituted by OH, C₁₋₆alkyl substituted by 1 to 3 substituents selected from the group consisting of halogen, C₃₋₈cycloalkyl, phenyl and phenyl substituted by OH, or CH₂—R_{4z} wherein R_{4z} is OH, acyloxy or a residue of formula (a)

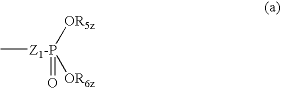

wherein Z₁ is a direct bond or O, preferably O;

each of R_{5z} and R_{6z}, independently, is H, or C₁₋₄alkyl optionally substituted by 1, 2 or 3 halogen atoms;

R_{1z} is OH, acyloxy or a residue of formula (a); and each of R_{2z} and R_{3z} independently, is H, C₁₋₄alkyl or acyl.

Group of formula X is a functional group attached as a terminal group to a moiety which may be hydrophilic or lipophilic and comprise one or more aliphatic, alicyclic, aromatic and/or heterocyclic residues, to the extent that the resulting molecule wherein at least one of Z and R_{1z} is or comprises a residue of formula (a), signals as an agonist at one of more sphingosine-1-phosphate receptor.

S1P receptor modulators or agonists are compounds which signal as agonists at one or more sphingosine-1 phosphate receptors, e.g. S1P1 to S1P8. Agonist binding to a S1P receptor may e.g. result in dissociation of intracellular heterotrimeric G-proteins into Gα-GTP and Gβγ-GTP, and/or increased phosphorylation of the agonist-occupied receptor and activation of downstream signaling pathways/kinases.

The binding affinity of S1P receptor agonists or modulators to individual human S1P receptors may be determined in following assay:

S1P receptor agonist or modulator activities of compounds are tested on the human S1P receptors S1P₁, S1P₂, S1P₃, S1P₄ and S1P₅. Functional receptor activation is assessed by quantifying compound induced GTP [γ-³⁵S] binding to membrane protein prepared from transfected CHO or RH7777 cells stably expressing the appropriate human S1P receptor. The assay technology used is SPA (scintillation proximity based assay). Briefly, DMSO dissolved compounds are serially diluted and added to SPA-bead (Amersham-Pharmacia) immobilised S1P receptor expressing membrane protein (10-20 μg/well) in the presence of 50 mM Hepes, 100 mM NaCl, 10 mM MgCl₂, 10 μM GDP, 0.1% fat free BSA and 0.2 nM GTP [γ-³⁵S] (1200 Ci/mmol). After incubation in 96 well microtiterplates at RT for 120 min, unbound GTP [γ³⁵S] is separated by a centrifugation step. Luminescence of SPA beads triggered by membrane bound GTP [γ³⁵S] is quantified with a TOPcount plate reader (Packard). EC₅₀s are calculated using standard curve fitting software. In this assay, the S1P receptor modulators or agonists preferably have a binding affinity to S1P receptor <50 nM.

Preferred S1P receptor agonists or modulators are e.g. compounds which in addition to their S1P binding properties also have accelerating lymphocyte homing properties, e.g. compounds which elicit a lymphopenia resulting from a re-distribution, preferably reversible, of lymphocytes from circulation to secondary lymphatic tissue, without evoking a generalized immunosuppression. Naïve cells are sequestered; CD4 and CD8 T-cells and B-cells from the blood are stimulated to migrate into lymph nodes (LN) and Peyer's patches (PP).

The lymphocyte homing property may be measured in following Blood Lymphocyte Depletion assay:

A S1P receptor agonist or modulator or the vehicle is administered orally by gavage to rats. Tail blood for hematological monitoring is obtained on day-1 to give the baseline individual values, and at 2, 6, 24, 48 and 72 hours after application. In this assay, the S1P receptor agonist or modulator depletes peripheral blood lymphocytes, e.g. by 50%, when administered at a dose of e.g. <20 mg/kg.

Examples of appropriate S1P receptor modulators or agonists are, for example:
Compounds as disclosed in EP627406A1, e.g. a compound of formula I

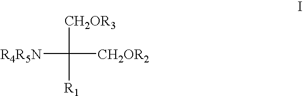

wherein R₁ is a straight- or branched (C₁₂₋₂₂)chain
which may have in the chain a bond or a hetero atom selected from a double bond, a triple bond, O, S, NR₆, wherein R₆ is H, C₁₋₄alkyl, aryl-C₁₋₄alkyl, acyl or (C₁₋₄alkoxy)carbonyl, and carbonyl, and/or
   which may have as a substituent C₁₋₄alkoxy, C₂₋₄alkenyloxy, C₂₋₄alkynyloxy, arylC₁₋₄alkyl-oxy, acyl, C₁₋₄alkylamino, C₁₋₄alkylthio, acylamino, (C₁₋₄alkoxy)carbonyl, (C₁₋₄alkoxy)-carbonylamino, acyloxy, (C₁₋₄alkyl)carbamoyl, nitro, halogen, amino, hydroxyimino, hydroxy or carboxy; or

R₁ is
a phenylalkyl wherein alkyl is a straight-or branched (C₆₋₂₀)carbon chain; or
a phenylalkyl wherein alkyl is a straight-or branched (C₁₋₃₀)carbon chain wherein said phenylalkyl is substituted by
a straight- or branched (C₆₋₂₀)carbon chain optionally substituted by halogen,
a straight- or branched (C₆₋₂₀)alkoxy chain optionally substitued by halogen,
a straight- or branched (C₆₋₂₀)alkenyloxy,
phenyl-C₁₋₁₄alkoxy, halophenyl-C₁₋₄alkoxy, phenyl-C₁₋₁₄alkoxy-C₁₋₁₄alkyl, phenoxy-C₁₋₄alkoxy or phenoxy-C₁₋₄alkyl,
cycloalkylalkyl substituted by C₆₋₂₀alkyl,
heteroarylalkyl substituted by C₆₋₂₀alkyl,
heterocyclic C₆₋₂₀alkyl or
heterocyclic alkyl substituted by C₂₋₂₀alkyl,

and wherein

the alkyl moiety may have
in the carbon chain, a bond or a heteroatom selected from a double bond, a triple bond, O, S, sulfinyl, sulfonyl, or NR₆, wherein R₆ is as defined above, and
as a substituent C₁₋₄alkoxy, C₂₋₄alkenyloxy, C₂₋₄alkynyloxy, arylC₁₋₄alkyloxy, acyl, C₁₋₄alkyl-amino, C₁₋₄alkylthio, acylamino, (C₁₋₄alkoxy)carbonyl, (C₁₋₄alkoxy)carbonylamino, acyloxy, (C₁₋₄alkyl)carbamoyl, nitro, halogen, amino, hydroxy or carboxy, and

each of R₂, R₃, R₄ and R₅, independently, is H, C₁₋₄ alkyl or acyl

or a pharmaceutically acceptable salt or hydrate thereof;
Compounds as disclosed in EP 1002792A1, e.g. a compound of formula II

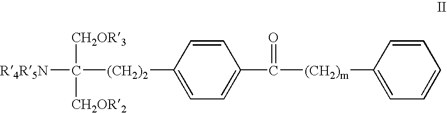

wherein m is 1 to 9 and each of R′₂, R′₃, R′₄ and R′₅, independently, is H, C₁₋₆alkyl or acyl, or a pharmaceutically acceptable salt or hydrate thereof;
Compounds as disclosed in EP0778263 A1, e.g. a compound of formula III

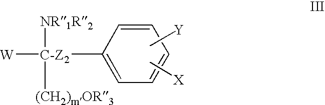

wherein W is H; C₁₋₆alkyl, C₂₋₆alkenyl or C₂₋₆alkynyl; unsubstituted or by OH substituted phenyl; R″₄O(CH₂)ₙ; or C₁₋₆alkyl substituted by 1 to 3 substituents selected from the group consisting of halogen, C₃₋₈cycloalkyl, phenyl and phenyl substituted by OH;

X is H or unsubstituted or substituted straight chain alkyl having a number p of carbon atoms or unsubstituted or substituted straight chain alkoxy having a number (p-1) of carbon atoms, e.g. substituted by 1 to 3 substitutents selected from the group consisting of C₁₋₆alkyl, OH, C₁₋₆alkoxy, acyloxy, amino, C₁₋₆alkylamino, acylamino, oxo, haloC₁₋₆alkyl, halogen, unsubstituted phenyl and phenyl substituted by 1 to 3 substituents selected from the group consisting of C₁₋₆alkyl, OH, C₁₋₆alkoxy, acyl, acyloxy, amino, C₁₋₆alkylamino, acylamino, haloC₁₋₆alkyl and halogen; Y is H, C₁₋₆alkyl, OH, C₁₋₆alkoxy, acyl, acyloxy, amino, C₁₋₆alkylamino, acylamino, haloC₁₋₆alkyl or halogen, Z₂ is a single bond or a straight chain alkylene having a number or carbon atoms of q,

each of p and q, independently, is an integer of 1 to 20, with the proviso of 6≦p+q≦23, m′ is 1, 2 or3, n is 2 or3,

each of R″₁, R″₂, R″₃ and R″₄, independently, is H, C₁₋₄alkyl or acyl, or a pharmaceutically acceptable salt or hydrate thereof,
Compounds as disclosed in WO02/18395, e.g. a compound of formula IVa or IVb

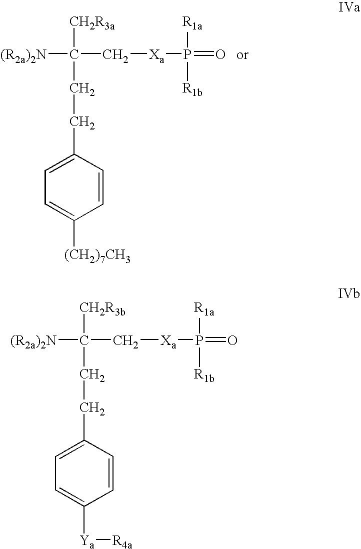

wherein Xₐ, is O, S, NR₁ₛ or a group —(CH₂)ₙₐ—, which group is unsubstituted or substituted by 1 to 4 halogen; nₐ is 1 or 2, R₁ₛ is H or (C₁₋₄)alkyl, which alkyl is unsubstituted or substituted by halogen; R₁ₐ is H, OH, (C₁₋₄)alkyl or O(C₁₋₄)alkyl wherein alkyl is unsubstituted or substituted by 1 to 3 halogen; R_{1b} is H, OH or (C₁₋₄)alkyl, wherein alkyl is unsubstituted or substituted by halogen; each R₂ₐ is independently selected from H or (C₁₋₄)alkyl, which alkyl is unsubstituted or substitued by halogen; R₃ₐ is H, OH, halogen or O(C₁₋₄)alkyl wherein alkyl is unsubstituted or substituted by halogen; and R_{3b} is H, OH, halogen, (C₁₋₄)alkyl wherein alkyl is unsubstituted or substituted by hydroxy, or O(C₁₋₄)alkyl wherein alkyl is unsubstituted or substituted by halogen; Yₐ is —CH₂—, —C(O)—, —CH(OH)—, —C(═NOH)—, O or S, and R₄ₐ is (C₄₋₁₄)alkyl or (C₄₋₁₄)alkenyl;

or a pharmaceutically acceptable salt or hydrate thereof;
Compounds as disclosed in WO02/06268AI, e.g. a compound of formula V

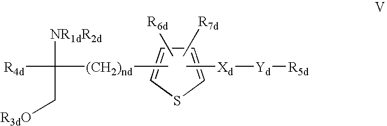

wherein each of R_{1d} and R_{2d}, independently, is H or an amino-protecting group;

R_{3d} is hydrogen, a hydroxy-protecting group or a residue of formula

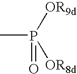

R_{4d} is C₁₋₄alkyl;

n_{d} is an integer of 1 to 6;

X_{d} is ethylene, vinylene, ethynylene, a group having a formula —D—CH₂— (wherein D is carbonyl, —CH(OH)—, O, S or N), aryl or aryl substituted by up to three substitutents selected from group a as defined hereinafter;

Y_{d} is single bond, C₁₋₁₀alkylene, C₁₋₁₀alkylene which is substituted by up to three substitutents selected from groups a and b, C₁₋₁₀alkylene having O or S in the middle or end of the carbon chain, or C₁₋₁₀alkylene having O or S in the middle or end of the carbon chain which is substituted by up to three substituents selected from groups a and b;

R_{5d} is hydrogen, C₃₋₆cycloalkyl, aryl, heterocyclic group, C₃₋₆cycloalkyl substituted by up to three substituents selected from groups a and b, aryl substituted by up to three substituents selected from groups a and b, or heterocyclic group substituted by up to three substituents selected from groups a and b;

each of R_{6d} and R_{7d}, independently, is H or a substituent selected from group a;

each of R_{8d} and R_{9d}, independently, is H or C₁₋₄alkyl optionally substituted by halogen;

<group a> is halogen, lower alkyl, halogeno lower alkyl, lower alkoxy, lower alkylthio, carboxyl, lower alkoxycarbonyl, hydroxy, lower aliphatic acyl, amino, mono-lower alkylamino, di-C₁₋₄alkylamino, acylamino, cyano or nitro; and

<group b> is C₃₋₆cycloalkyl, aryl or heterocyclic group, each being optionally substituted by up to three substituents selected from group a;

with the proviso that when R_{5d} is hydrogen, Y_{d} is a either a single bond or linear C₁₋₁₀ alkylene, or a pharmacologically acceptable salt, ester or hydrate thereof;
as disclosed in JP-14316985 (JP2002316985), e.g. a compound of formula VI

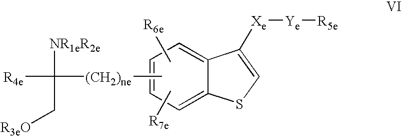

wherein R₁ₑ, R₂ₑ, R₃ₑ, R₄ₑ, R₅ₑ, R₆ₑ, R₇ₑ, nₑ, Xₑ and Yₑ are as disclosed in JP-14316985;

or a pharmacologically acceptable salt, ester or hydrate thereof;
Compounds as disclosed in WO 03/29184 and WO 03/29205, e.g. compounds of formula IX

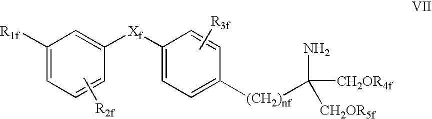

wherein X_{f} is O, S, SO or SO₂

R_{1f} is halogen, trihalomethyl, OH, C₁₋₇alkyl, C₁₋₄alkoxy, trifluoromethoxy, phenoxy, cyclohexylmethyloxy, pyridylmethoxy, cinnamyloxy, naphthylmethoxy, phenoxymethyl, CH₂—OH, CH₂—CH₂—OH, C₁₋₄alkylthio, C₁₋₄alkylsulfinyl, C₁₋₄alkylsulfonyl, benzylthio, acetyl, nitro or cyano, or phenyl, phenylC₁₋₄alkyl or phenyl—C₁₋₄alkoxy each phenyl group thereof being optionally substituted by halogen, CF₃, C₁₋₄alkyl or C₁₋₄alkoxy;

R_{2f} is H, halogen, trihalomethyl, C₁₋₄alkoxy, C₁₋₇alkyl, phenethyl or benzyloxy;

R_{3f} H, halogen, CF₃, OH, C₁₋₇alkyl, C₁₋₄alkoxy, benzyloxy or C₁₋₄alkoxymethyl;

each of R_{4f} and R_{5f}, independently is H or a residue of formula

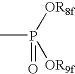

wherein each of R_{8f} and R_{9f}, independently, is H or C₁₋₄alkyl optionally substituted by halogen; and

n_{f} is an integer from 1 to 4;

or a pharmacological salt, solvate or hydrate thereof;
Compounds as disclosed in WO03/062252A1, e.g. a compound of formula VIII

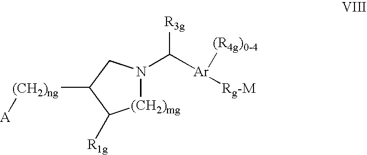

wherein

Ar is phenyl or naphthyl; each of m_{g} and n_{g} independently is 0 or 1; A is selected from COOH, PO₃H₂, PO₂H, SO₃H, PO(C₁₋₃alkyl)OH and 1H-tetrazol-5-yl; each of R_{1g} and R_{2g} independently is H, halogen, OH, COOH or C₁₋₄alkyl optionally substituted by halogen; R_{3g} is H or C₁₋₄alkyl optionally substituted by halogen or OH; each R_{4g} independently is halogen, or optionally halogen substituted C₁₋₄alkyl or C₁₋₃alkoxy; and each of R_{g} and M has one of the significances as indicated for B and C, respectively, in WO03/062252A1;

or a pharmacologically acceptable salt, solvate or hydrate thereof;
Compounds as disclosed in WO 03/062248A2, e.g. a compound of formula IX

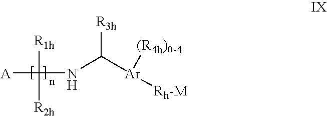

wherein Ar is phenyl or naphthyl; n is 2,3 or 4; A is COOH, 1H-tetrazol-5-yl, PO₃H₂, PO₂H₂, —SO₃H or PO(R₅ₕ)OH wherein R₅ₕ is selected from C₁₋₄alkyl, hydroxyC₁₋₄alkyl, phenyl, —CO—C₁₋₃alkoxy and —CH(OH)-phenyl wherein said phenyl or phenyl moiety is optionally substituted;

each of R₁ₕ and R₂ₕ independently is H, halogen, OH, COOH, or optionally halogeno substituted C₁₋₆alkyl or phenyl; R₃ₕ is H or C₁₋₄alkyl optionally substituted by halogen and/OH; each R₄ₕ independently is halogeno, OH, COOH, C₁₋₄alkyl, S(O)_{0, 1 or2}C₁₋₃alkyl, C₁₋₃alkoxy, C₃₋₆cycloalkoxy, aryl or aralkoxy, wherein the alkyl portions may optionally be substituted by 1-3 halogens; and each of Rₕ and M has one of the significances as indicated for B and C, respectively, in WO03/062248A2

or a pharmacologically acceptable salt, solvate or hydrate thereof.
Compounds as disclosed in WO 04/026817A, e.g. compounds of formula X

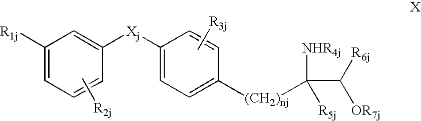

wherein

R₁ⱼ is halogen, trihalomethyl, C₁₋₄alkyl, C₁₋₄alkoxy, C₁₋₄alkylthio, C₁₋₄alkylsulifinyl, C₁₋₄alkyl-sulfonyl aralkyl, optionally substituted phenoxy or aralkyloxy, R₂ⱼ is H, halogen, trihalo-methyl, C₁₋₄alkyl, C₁₋₄alkoxy, aralkyl or aralkyloxy, R₃ⱼ is H, halogen, CF₃, C₁₋₄alkyl, C₁₋₄alkoxy, C₁₋₄alkylthio or benzyloxy, R₄ⱼ is H, C₁₋₄alkyl, phenyl, optionally substituted benzyl or benzoyl, or lower aliphatic C₁₋₅acyl, R₅ⱼ is H, monohalomethyl, C₁₋₄alkyl, C₁₋₄alkoxymethyl, C₁₋₄alkyl-thiomethyl, hydroxyethyl, hydroxypropyl, phenyl, aralkyl, C₂₋₄alkenyl or -alkynyl, each of R₆ⱼ and R₇ⱼ, independently, is H or C₁₋₄alkyl, Xⱼ is O, S, SO or SO₂ and nⱼ is an integer of 1 to 4, or a pharmacologically acceptable salt, solvate or hydrate thereof.
Compounds as disclosed in WO 04/103306A, WO 05/000833, WO 05/103309 or WO 05/113330, e.g. compounds of formula XIa or XIb

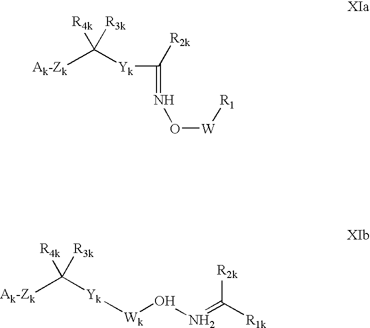

wherein

Aₖ is COOR₅ₖ, OPO(OR₅ₖ)₂, PO(OR₅ₖ)₂, SO₂OR₅ₖ, POR₅ₖOR₅ₖ or 1H-tetrazol-5yl, R₅ₖ being H or C₁₋₆alkyl;

Wₖ is a bond, C₁₋₃alkylene or C₂₋₃alkenylene;

Yₖ is C₆₋₁₀aryl or C₃₋₉heteroaryl, optionally substituted by 1 to 3 radicals selected from halogene, OH, NO₂, C₁₋₆alkyl, C₁₋₆alkoxy; halo-substituted C₁₋₆alkyl and halo-substituted C₁₋₆alkoxy;

Zₖ is a heterocyclic group as indicated in WO 04/103306A, e.g. azetidine;

R₁ₖ is C₆₋₁₀aryl or C₃₋₉heteroaryl, optionally substituted by C₁₋₆alkyl, C₆₋₁₀aryl, C₆₋₁₀arylC₁₋₄alkyl, C₃₋₉heteroaryl, C₃₋₉heteroarylC₁₋₄alkyl, C₃₋₈cycloalkyl, C₃₋₈cycloalkylC₁₋₄alkyl, C₃₋₈heterocycloalkyl or C₃₋₈heterocycloalkylC₁₋₄alkyl; wherein any aryl, heteroaryl, cycloalkyl or heterocycloalkyl of R₁ₖ may be substituted by 1 to 5 groups selected from halogen, C₁₋₆alkyl, C₁₋₆alkoxy and halo substituted-C₁₋₆alkyl or -C₁₋₆alkoxy;

R₂ₖ is H, C₁₋₆alkyl, halo substituted C₁₋₆alkyl, C₂₋₆alkenyl or C₂₋₆alkynyl: and

each of R₃ₖ or R₄ₖ, independently, is H, halogen, OH, C₁₋₆alkyl, C₁₋₆alkoxy or halo substituted C₁₋₆alkyl or C₁₋₆alkoxy;

and the N-oxide derivatives thereof or prodrugs thereof,

or a pharmacologically acceptable salt, solvate or hydrate thereof.

The compounds of formulae I to XIb may exist in free or salt form. Examples of pharmaceutically acceptable salts of the compounds of the formulae I to VI include salts with inorganic acids, such as hydrochloride, hydrobromide and sulfate, salts with organic acids, such as acetate, fumarate, maleate, benzoate, citrate, malate, methanesulfonate and benzenesulfonate salts, or, when appropriate, salts with metals such as sodium, potassium, calcium and aluminium, salts with amines, such as triethylamine and salts with dibasic amino acids, such as lysine. Examples of pharmaceutically acceptable salts of the compounds of the formulae VII and X include salts with inorganic acids, such as hydrochloride and hydrobromide, salts with organic acids, such as acetate, trifluoroacetate, citrate, tartrate, methanesulfonate and benzenesulfonate salts. The compounds and salts of the combination of the present invention encompass hydrate and solvate forms.

Acyl as indicated above may be a residue R_{Y}—CO— wherein R_{y} is C₁₋₆alkyl, C₃₋₆cycloalkyl, phenyl or phenyl-C₁₋₄alkyl. Unless otherwise stated, alkyl, alkoxy, alkenyl or alkynyl may be straight or branched.

Aryl may be phenyl or naphthyl, preferably phenyl.

When in the compounds of formula I the carbon chain as R₁ is substituted, it is preferably substituted by halogen, nitro, amino, hydroxy or carboxy. When the carbon chain is interrupted by an optionally substituted phenylene, the carbon chain is preferably unsubstituted. When the phenylene moiety is substituted, it is preferably substituted by halogen, nitro, amino, methoxy, hydroxy or carboxy.

Preferred compounds of formula I are those wherein R₁ is C₁₃₋₂₀alkyl, optionally substituted by nitro, halogen, amino, hydroxy or carboxy, and, more preferably those wherein R₁ is phenylalkyl substituted by C₆₋₁₄-alkyl chain optionally substituted by halogen and the alkyl moiety is a C₁₋₆alkyl optionally substituted by hydroxy. More preferably, R₁ is phenyl-C₁₋₆alkyl substituted on the phenyl by a straight or branched, preferably straight, C₆₋₁₄alkyl chain. The C₆₋₁₄alkyl chain may be in ortho, meta or para, preferably in para.

Preferably each of R₂ to R₅ is H.

In the above formula of V “heterocyclic group” represents a 5-to 7 membered heterocyclic group having 1 to 3 heteroatoms selected from S, O and N. Examples of such heterocyclic groups include the heteroaryl groups indicated above, and heterocyclic compounds corresponding to partially or completely hydrogenated heteroaryl groups, e.g. furyl, thienyl, pyrrolyl, azepinyl, pyrazolyl, imidazolyl, oxazolyl, isoxazolyl, thiazolyl, isothiazolyl, 1,2,3-oxadiazolyl, triazolyl, tetrazolyl, thiadiazolyl, pyranyl, pyridyl, pyridazinyl, pyrimidinyl, pyrazinyl, tetrahydropyranyl, morpholinyl, thiomorpholinyl, pyrrolidinyl, pyrrolyl, imidazolidinyl, pyrazolidinyl, piperidinyl, piperazinyl, oxazolidinyl, isoxazolidinyl, thiazolidinyl or pyrazolidinyl. Preferred heterocyclic groups are 5-or 6-membered heteroaryl groups and the most preferred heteocyclic group is a morpholinyl, thiomorpholinyl or piperidinyl group.

A preferred compound of formula I is 2-amino-2-tetradecyl-1,3-propanediol. A particularly preferred S1P receptor agonist of formula I is FTY720, i.e. 2-amino-2-[2-(4-octylphenyl) ethyl]propane-1,3-diol in free form or in a pharmaceutically acceptable salt form (referred to hereinafter as Compound A), e.g. the hydrochloride, as shown:

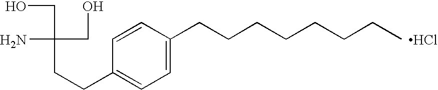

A preferred compound of formula II is the one wherein each of R′₂ to R′₅ is H and m is 4, i.e. 2-amino-2-{2-[4-(1-oxo5-phenylpentyl)phenyl]ethyl}propane-1,3-diol, in free form or in pharmaceutically acceptable salt form (referred to hereinafter as Compound B), e.g the hydrochloride.

A preferred compound of formula III is the one wherein W is CH₃, each of R″₁, to R″₃ is H, Z₂ is ethylene, X is heptyloxy and Y is H, i.e. 2-amino-4-(4-heptyloxyphenyl)-2-methyl-butanol, in free form or in pharmaceutically acceptable salt form (referred to hereinafter as Compound C), e.g. the hydrochloride. The R-enantiomer is particularly preferred.

A preferred compound of formula IVa is the FTY720-phosphate (R₂ₐ is H, R₃ₐ is OH, Xₐ is O, R₁ₐ and R_{1b} are OH). A preferred compound of formula IVb is the Compound C-phosphate (R₂ₐ is H, R_{3b} is OH, Xₐ is O, R₁ₐ and R_{1b} are OH, Yₐ is O and R₄ₐ is heptyl). A preferred compound of formula V is Compound B-phosphate.

A preferred compound of formula VI is (2R)-2-amino-4-[3-(4-cyclohexyloxybutyl)-benzo[b]thien-6-yl]-2-methylbutan-1 -ol.

A preferred compound of formula VII is e.g. 2-amino-2-[4-(3-benzyloxyphenoxy)-2-chlorophenyl]ethyl-1,3-propane-diol, 2-amino-2-[4-(benzyloxyphenylthio)-2-chlorophenyl]ethyl-1,3-propane-diol, 2-amino-2-[4-(3-benzyloxyphenoxy)-2-chlorophenyl]propyl-1,3-propane-diol or 2-amino-2-[4-(benzyloxyphenylthio)-2-chlorophenyl]propyl-1,3-propane-diol.

A preferred compound of formula X is e.g. 2-amino-4-[4-(3-benzyloxyphenylthio)-2-chlorophenyl]-2-methylbutane-1-ol or 2-amino-4-[4-(3-benzyloxyphenylthio)-2-chlorophenyl]-2-ethylbutane-1-ol.

A preferred compound of formula XIa is e.g. 1-{4-[1-(4-cyclohexyl-3-trifluoromethyl-benzyloxyimino)-ethyl]-2-ethyl-benzyl}-azetidine-3-carboxylic acid, or a prodrug thereof.

The various known S1P receptor agonists show structural similarities, which result in related problems in providing a suitable formulation for oral administration.

There is a need for a S1P receptor modulator or agonist containing oral formulation which can easily be swallowed, e.g. by children, patients in a difficult condition to swallow due to their diseases or older patients. A liquid dosage is especially preferred for such patients because of the ease with which it may be swallowed and thus patients may be more inclined to comply with their medication instruction. Additionally, a liquid formulation provides flexibility in mg/kg dosing.

However, compositions in form of aqueous solutions comprising a S1P receptor modulator or agonist or a pharmaceutically acceptable salt thereof have been observed to lead to crystalline deposits of the drug either shortly after preparation or upon storage.

Furthermore, when formulating a drug for oral administration to pediatric patients, one is limited to a smaller number of suitable excipients, e.g. such a composition should preferably be ethanol-free.

It has now been found that compositions in form of a concentrate for dilution comprising propylene glycol and optionally glycerin are physically stable for extended periods of time, e.g. more than six months at ambient temperature.

Accordingly, the present application provides a concentrate for dilution comprising a S1P receptor modulator or agonist or a pharmaceutically acceptable salt thereof, propylene glycol and optionally glycerin.

The concentrate for dilution of the invention preferably contains about 5 to 20% by weight of S1P receptor agonists, more preferably about 7 to 15%, e.g. about 10% by weight, based on the total weight of the composition.

The amount of glycerin in the concentrate for dilution of the invention typically ranges from 0 to 35%, e.g.1 to 35%, e.g. about 5 to 25% by weight, based on the total weight of the composition.

The amount of propylene glycol in the concentrate for dilution of the invention typically ranges from about 65 to 100%, e.g. about 65 to 99%, e.g. 75 to 95% by weight, based on the total weight of the composition.

The ratio of glycerin (when present) to propylene glycol in the concentrate for dilution of the invention typically is between about 5:95 to about 25:75, e.g. about 5:95, 10:90, 15:85, 20:80, 25:75, preferably about 25:75.

Preferably, the concentrate for dilution of the invention shows a flow behavior to allow dosing with a syringe.

The concentrate for dilution of the invention may comprise one or more further excipients e.g. yet another solvent, a flavor and/or a preservative.

Preferably, the concentrates of the present invention are ethanol-free.

Suitable flavor include citrus flavors including cherry, strawberry, grape, punch, tutti-frutti, e.g. as available from Firmenich Inc. The amount of flavor in the concentrate for dilution of the invention ranges from 0 to 0.5% by weight, based on the total weight of the composition.

Suitable preservative include a hydroxybenzoic acid derivative, e.g. methyl-, propyl- or butyl-paraben. The amount of preservative in the concentrate for dilution of the invention ranges from 0.05 to 0.13% by weight, based on the total weight of the composition.

The concentrate for dilution of the invention may be produced by standard processes, for instance by conventional mixing. Procedures which may be used are known in the art, e.g. those described in L. Lachman et al. The Theory and Practice of Industrial Pharmacy, 3^{rd} Ed, 1986, H. Sucker et al, Pharmazeutische Technologie, Thieme, 1991, Hagers Handbuch der pharmazeutischen Praxis, 4^{th} Ed. (Springer Verlag, 1971) and Remington's Pharmaceutical Sciences, 13^{th} Ed., (Mack Publ., Co., 1970) or later editions.

In one aspect, the present invention relates to a process for producing a concentrate of the invention comprising dissolution of a S1P receptor modulator or agonist or a pharmaceutically acceptable salt thereof and, optionally, another solvent, a flavor and/or a preservative, in a propylene glycol and addition of glycerin.

Prior to administration, the required amount of concentrate of the invention is dosed e.g. with a syringe, and diluted with a vehicle.

Suitable dilution vehicles include water, sparkling water, fruit juices e.g. orange or apple juice, soda such as colas, limeade, and lemonade.

The ratio of concentrate to dilution vehicle may be from 1:1 to more than 1:10; preferably it is more than 1:10.

In another aspect, the present invention also provides a pharmaceutical kit comprising the concentrate and the dilution vehicle.

The pharmaceutical solution so formed may be preferably used immediately or within a short time of being formed, e.g. within four hours.

The concentrates of the present invention or the pharmaceutical solutions resulting from the dilution are useful, either alone or in combination with other active agents, for the treatment and prevention of conditions e.g. as disclosed in U.S. Pat. No. 5,604,229, WO 97/24112, WO 01/01978, U.S. Pat. No. 6,004,565, U.S. Pat. No. 6,274,629 and JP-14316985 for the compounds of formula I, e.g. in WO 03/29184 and WO 03/29205 for the compounds of formula VII, in WO 04/026817A for the compounds of formula X, or in WO 04/103306A, WO 05/000833, WO 05/103309 or WO 05/113330 for the compounds of formulae XIa and XIb, the contents of which are incorporated herein by reference.

In particular, a concentrate of the invention or the pharmaceutical solution resulting from the dilution is useful for:
a) treatment and prevention of organ or tissue transplant rejection, for example for the treatment of the recipients of heart, lung, combined heart-lung, liver, kidney, pancreatic, skin or corneal transplants, and the prevention of graft-versus-host disease, such as sometimes occurs following bone marrow transplantation; particularly in the treatment of acute or chronic allo-and xenograft rejection or in the transplantation of insulin producing cells, e.g. pancreatic islet cells;
b) treatment and prevention of autoimmune disease or of inflammatory conditions, e.g. multiple sclerosis, arthritis (for example rheumatoid arthritis), inflammatory bowel diseases, hepatitis, etc.;
c) treatment and prevention of viral myocarditis and viral diseases caused by viral myocarditis, including hepatitis and AIDS.

The concentrate for dilution or the pharmaceutical solution made therefrom, may be administered to a patient in need of immunosuppression, in an amount which is therapeutically effective, e.g. against a disease or condition which can be treated by administration of the S1P receptor modulator or agonist. The exact amount of S1P receptor modulator or agonist or pharmaceutically acceptable salt thereof to administer can vary widely. The dose may depend on the particular compound, the rate of administration, the strength of the particular concentrate or pharmaceutical solution employed, the nature of the disease or condition being treated, and the sex, age and body weight of the patient. The dose may also depend on the existence, nature and extent of any adverse side-effects that may accompany the administration of the concentrate or pharmaceutical formulation. Typically, a dose of 0.5 to 5 mg of S1P receptor modulator or agonist, e.g. Compound A, may be administered to a child or an adult patient having difficulties to swallow.

The concentrate for dilution or the respective pharmaceutical solution may be used in combination with other immunosuppressant(s), steroid(s) such as prednisolone, methylprednisolone, dexamethasone, hydrocortisone and the like, or nonsteroidal anti-inflammatory agent. The administration of a combination of active agents may be simultaneous or consecutive, with either one of the active agents being administered first. The dosage of the active agents of a combination treatment may depend on effectiveness and site of action of each active agent, as well as synergistic effects between the agents used for combination therapy.

A preferred concentrate or pharmaceutical solution for oral administration, is the one comprising Compound A hydrochloride, as S1P receptor modulator, e.g. for use in the treatment of multiple sclerosis.

The invention will now be described with reference to the following specific embodiments, without any limitation.

### EXAMPLES 1 TO 3

| | Ex. 1 | | Ex. 2 | | Ex. 3 | |
|---|---|---|---|---|---|---|
| Compound A hydrochloride | 11.12 | mg | 11.12 | mg | 5.56 | mg |
| Glycerin | 250 | mg | 10 | mg | 350 | mg |
| Methyl paraben | — | | 0.5 | mg | — | |
| Tutti Frutti Flavor | 2.5 | mg | 5 | mg | 2.5 | mg |
| Propylene glycol | qsad 1 | ml | qsad 1 | ml | qsad 1 | ml |

Compound A, the flavor and the preservative, if present, are dissolved in propylene glycol in amounts given in the table. Then glycerin (in an amount as given in the table) is added to said solution.

The composition is stable for at least six months at ambient temperature.

Prior to administration, about 0.05 ml to 1 ml of the concentrate are diluted with about 10 ml or more of water, fruit juice or soda.

By following the procedure as described above, the following compositions may be prepared:

### EXAMPLES 4 TO 6

| | Ex. 4 | | Ex. 5 | | Ex. 6 | |
|---|---|---|---|---|---|---|
| Compound A hydrochloride | 2.78 | mg | 2.78 | mg | 1.39 | mg |
| Glycerin | 250 | mg | 10 | mg | 350 | mg |
| Methyl paraben | — | | 0.5 | mg | — | |
| Tutti Frutti Flavor | 2.5 | mg | 5 | mg | 2.5 | mg |
| Propylene glycol | qsad 1 | ml | qsad 1 | ml | qsad 1 | ml |

The same formulations as disclosed above may be prepared without flavor or with another flavor.

The same formulations as disclosed above may be prepared without glycerin.

1. A concentrate for dilution comprising a S1P receptor modulator or agonist or a pharmaceutically acceptable salt thereof, and propylene glycol.

2. A concentrate according to claim 1, comprising in addition glycerin.

3. A concentrate according to claim 1, wherein the S1P receptor modulator or agonist is a compound comprising a group of formula X 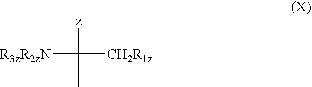
wherein Z is H, C₁₋₆alkyl, C₂₋₆alkenyl, C₂₋₆alkynyl, phenyl, phenyl substituted by OH, C₁₋₆alkyl substituted by 1 to 3 substituents selected from the group consisting of halogen, C₃₋₈cycloalkyl, phenyl and phenyl substituted by OH, or CH₂—R_{4z} wherein R_{4z} is OH, acyloxy or a residue of formula (a) 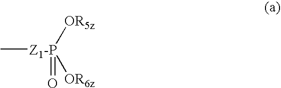
wherein Z₁ is a direct bond or O, preferably O;
each of R_{5z} and R_{6z}, independently, is H, or C₁₋₄alkyl optionally substituted by 1, 2 or 3 halogen atoms;
R_{1z} is OH, acyloxy or a residue of formula (a); and each of R_{2z} and R_{3z} independently, is H, C₁₋₄alkyl or acyl.
or a pharmaceutically acceptable salt thereof.

4. A concentrate according to claim 1 comprising a S1P receptor modulator or agonist selected from 2-amino-2-[2-(4-octylphenylethyl)]propane-1,3-diol, 2-amino-2-[4-(benzyloxyphenylthio)-2-chlorophenyl]ethyl-1,3-propane-diol, or a corresponding phosphate thereof, and 1-{4-[1-(4-cyclohexyl-3-trifluoromethyl-benzyloxyimino)-ethyl]-2-ethyl-benzyl}-azetidine-3-carboxylic acid, or a pharmaceutically acceptable salt thereof.

5. A concentrate according to claim 2 comprising glycerin and propylene glycol in a ratio of about 5:95 to about 25:75.

6. A concentrate according to claim 1 which is diluted with a vehicle in a ratio of from 1:1 to more than 1:10 prior to administration.

7. A pharmaceutical solution comprising a concentrate according to claim 1 diluted with a vehicle in a ratio of from 1:1 to more than 1:10.

8. A pharmaceutical solution according to claim 7 for oral administration.

9. (canceled)

10. A method of treating a subject in need of immunosuppression, comprising administering to the subject a concentrate according to claim 1 which is diluted with a vehicle in a ratio of from 1:1 to more than 1:10 prior to administration.
